SEQUENCE LISTING (23 sequences)

SEQ ID NO: 1 - DNA, 208 bp, synthetic construct
  misc_feature 1..208: note = Figwort subgenomic transcript promoter (-270 to -63 position from the transcription start position)
  source 1..208: mol_type = other DNA
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatga

SEQ ID NO: 2 - DNA, 181 bp, synthetic construct
  misc_feature 1..181: note = Mirabilis mosaic virus subgenomic transcript promoter (-306 to -125 position from the transcription start position)
  source 1..181: mol_type = other DNA
gttttacagtcaggacagataatgtaaatcttttaaaaggatttatgaataaaaagattactggtgacagtaaacagggaaggctaataagatggcaaatgtggttttcacattacacctttaaggtggaccacctaaaaggagaacaaaatgtgctggctgattatctcaccagagaatt

SEQ ID NO: 3 - DNA, 256 bp, synthetic construct
  misc_feature 1..256: note = Mirabilis mosaic virus full-length transcript promoter (-193 to ⊖position from the transcription start position)
  source 1..256: mol_type = other DNA
aaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatct

SEQ ID NO: 4 - DNA, 770 bp, synthetic construct
  misc_feature 1..770: note = FMM-UD promoter
  source 1..770: mol_type = other DNA
atccacgggtgacagccctccgacgggtgacagccctccgacgggtgacagccctccgacgggtgacagccctccggattttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgagttttacagtcaggacagataatgtaaatcttttaaaaggatttatgaataaaaagattactggtgacagtaaacagggaaggctaataagatggcaaatgtggttttcacattacacctttaaggtggaccacctaaaaggagaacaaaatgtgctggctgattatctcaccagagaattagcgtgggcggcgtgggcgtagatctagcgtgggcggcgtgggcgtaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatct

SEQ ID NO: 5 - DNA, 21 bp, synthetic construct
  misc_feature 1..21: note = 5'UTR
  source 1..21: mol_type = other DNA
attattacatcaaaacaaaaa

SEQ ID NO: 6 - DNA, 211 bp, synthetic construct
  misc_feature 1..211: note = CaMV 35S terminator
  source 1..211: mol_type = other DNA
gtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgac

SEQ ID NO: 7 - DNA, 556 bp, synthetic construct
  misc_feature 1..556: note = PI-II terminator
  source 1..556: mol_type = other DNA
ctagagtcaccctgcaatgtgaccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgatt

SEQ ID NO: 8 - DNA, 462 bp, synthetic construct
  misc_feature 1..462: note = RB7
  source 1..462: mol_type = other DNA
accaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 9 - DNA, 1242 bp, synthetic construct
  misc_feature 1..1242: note = 3PR terminator
  source 1..1242: mol_type = other DNA
gtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgagctcctagagtcaccctgcaatgtgaccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattgagctcaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 10 - DNA, 272 bp, synthetic construct
  misc_feature 1..272: note = BiP
  source 1..272: mol_type = other DNA
atggctcgctcgtttggagctaacagtaccgttgtgttggcgatcatcttcttcggtgagtgattttccgatcttcttctccgatttagatctcctctacattgttgcttaatctcagaaccttttttcgttgttcctggatctgaatgtgtttgtttgcaatttcacgatcttaaaaggttagatctcgattggtattgacgattggaatctttacgatttcaggatgtttatttgcgttgtcctctgcaatagaagaggctacgaagtta

SEQ ID NO: 11 - protein, 34 aa, synthetic construct
  REGION 1..34: note = BiP
MARSFGANSTVVLAIIFFGCLFALSSAIEEATKL

SEQ ID NO: 12 - DNA, 180 bp, synthetic construct
  misc_feature 1..180: note = MP
  source 1..180: mol_type = other DNA
gcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgat

SEQ ID NO: 13 - protein, 60 aa, synthetic construct
  REGION 1..60: note = MP
ANITVDYLYNKETKLFTAKLNVNENVECGNNTCTNNEVHNLTECKNASVSISHNSCTAPD

SEQ ID NO: 14 - DNA, 27 bp, synthetic construct
  misc_feature 1..27: note = linker
  source 1..27: mol_type = other DNA
ggtggaggtgggtctggtggtggatca

SEQ ID NO: 15 - protein, 9 aa, synthetic construct
  REGION 1..9: note = linker
GGGGSGGGS

SEQ ID NO: 16 - DNA, 480 bp, synthetic construct
  misc_feature 1..480: note = CBM3
  source 1..480: mol_type = other DNA
gtatcaggtaaccttaaggtggagttttacaactcgaacccttctgatacaactaactcaataaacccacagttcaaagttacaaacacaggcagctctgcgatcgatttgtctaaattaaccctcagatactattatacggttgatggacagaaggaccagactttctggtgtgatcatgcagctatcattggttctaacggtagctacaacggaattacatcaaacgtgaagggcactttcgttaagatgtcctctagcactaacaacgcagacacatatttggagatcagttttacggggggaacccttgaaccaggtgctcacgtccagattcaaggaagattcgctaaaaacgactggtcgaactatacccaaagtaatgattacagttttaaatccgcctcacaatttgttgagtgggatcaggtcactgcttacctgaacggggttctagtgtggggaaaggaacctggtccc

SEQ ID NO: 17 - protein, 160 aa, synthetic construct
  REGION 1..160: note = CBM3
VSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGP

SEQ ID NO: 18 - DNA, 234 bp, synthetic construct
  misc_feature 1..234: note = bdSUMO
  source 1..234: mol_type = other DNA
atgcatattaatttgaaagtgaagggacaggacggaaacgaggttttcttccgtatcaaaagatcaacacaactcaagaaactcatgaatgcttactgcgatagacaaagcgtggacatgacagctatagctttcctatttgatgggagaaggttgagagcggaacagactccggatgagcttgaaatggaagatggagatgagattgacgcaatgttacatcagactggtggt

SEQ ID NO: 19 - protein, 78 aa, synthetic construct
  REGION 1..78: note = bdSUMO
MHINLKVKGQDGNEVFFRIKRSTQLKKLMNAYCDRQSVDMTAIAFLFDGRRLRAEQTPDELEMEDGDEIDAMLHQTGG

SEQ ID NO: 20 - DNA, 552 bp, synthetic construct
  misc_feature 1..552: note = hIL6
  source 1..552: mol_type = other DNA
atggttcctccaggagaggattctaaggatgtggctgcacctcatagacagccattgacctcttcagaaagaatcgataagcaaattaggtacattctcgatggtatatctgctttaaggaaggagacatgtaataagtcaaacatgtgcgaaagttctaaggaggctctcgcagaaaataaccttaatttgcctaagatggctgaaaaagatggatgttttcagtctggtttcaacgaagagacttgccttgttaaaattatcacaggacttttggaatttgaggtgtatcttgaatacttacaaaacagattcgaatcaagtgaagagcaggctagggcagttcaaatgagtactaaggtgcttatacagttcttgcaaaagaaagctaagaaccttgatgcaatcactacacctgatccaaccactaatgcttcactcttaacaaagcttcaagcacagaaccaatggttgcaggatatgacaacccaccttattctcaggtcattcaaggagtttttacagtcaagtcttagggctcttaggcagatg

SEQ ID NO: 21 - protein, 184 aa, synthetic construct
  REGION 1..184: note = hIL6
MVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 22 - DNA, 12 bp, synthetic construct
  misc_feature 1..12: note = HDEL
  source 1..12: mol_type = other DNA
cacgatgagctc

SEQ ID NO: 23 - protein, 4 aa, synthetic construct
  REGION 1..4: note = HDEL
HDEL

(19) United States
(12) Patent Application Publication
(10) Pub. No.: US 2025/0243502 A1
(43) Pub. Date: Jul. 31, 2025

(54) HIGH EXPRESSION VECTOR INCLUDING STRONG RECOMBINANT PROMOTER AND/OR TERMINATOR FOR MASS-PRODUCING PROTEIN OF INTEREST IN PLANT, AND METHOD FOR MASS-PRODUCING PROTEIN OF INTEREST USING SAME

(71) Applicant: POSTECH RESEARCH AND BUSINESS DEVELOPMENT FOUNDATION, Pohang-si, Gyeongsangbuk-do (KR)

(72) Inventors: Inhwan HWANG, Pohang-si (KR); Joohyun KANG, Pohang-si (KR); A Reum YUN, Pohang-si (KR)

(73) Assignee: POSTECH RESEARCH AND BUSINESS DEVELOPMENT FOUNDATION, Pohang-si, Gyeongsangbuk-do (KR)

(21) Appl. No.: 18/845,991

(22) PCT Filed: Feb. 27, 2023
(86) PCT No.: PCT/KR2023/002727

§ 371 Date: Sep. 11, 2024

(30) Foreign Application Priority Data
Mar. 16, 2022 (KR) 10-2022-0032842

(51) Int. Cl. C12N 15/82

(52) U.S. Cl. CPC C12N 15/8257; C12N 15/8216

(57) ABSTRACT

The present invention relates to a high expression vector, including a strong recombinant promoter and a terminator, for mass-producing a protein of interest in a plant, and a method for mass-producing a protein of interest using the same. More specifically, the present invention provides a method for mass-producing a protein of interest in a plant by increasing the expression of the protein of interest by increasing the transcription level of the protein of interest by using a super promoter, produced by recombining strong promoter sites derived from plant viruses, and/or a strong terminator, produced by repeatedly connecting two types of terminators.

### TECHNICAL FIELD

The present invention relates to a high expression vector, including a strong recombinant promoter and a terminator, for mass-producing a protein of interest in a plant, and a method for mass-producing a protein of interest using the same. More specifically, the present invention provides a method for mass-producing a protein of interest in a plant by increasing the expression of the protein of interest by increasing the transcription level of the protein of interest by using a super promoter, produced by recombining strong promoter sites derived from plant viruses, and/or a strong terminator, produced by repeatedly connecting two types of terminators.

### BACKGROUND ART

Technologies for producing recombinant proteins from various living organisms have been developed. Recently, there have been attempts to mass-produce and commercialize useful recombinant proteins by using plants. It has been suggested that mass production of recombinant proteins using plants has several advantages. First of all, it was proposed that in terms of mass production, a much larger amount of protein could be produced at a lower cost than animal cells, E. coli and microorganisms. In addition, there are almost no toxins such as endotoxin present in E. coli or microorganisms in plants, and there are no pathogens in plants that can infect the human body. However, there are not many cases where recombinant proteins have been produced in plants and commercialized yet. The most important factor is that the level of gene expression in plant cells is not yet high, and thus, protein production using plants is less competitive than other systems. Therefore, there is an urgent need to develop a technique for a high expression system of genes that can mass-produce large quantities of proteins of interest in plants.

A terminator regulates the level of transcription by coordinating the termination of transcription and processing of the 3′ end of the mRNA. Accordingly, a strong terminator can improve the expression of foreign genes by several times or tens of times [Ingelbrecht et al., (1989) Different 3′ end regions strongly influence the level of gene expression in plant cells, Plant C. 1:671-680; Nagaya et al., (2010) The HSP Terminator of Arabidopsis thaliana Increases Gene Expression in Plant Cells. Plant Cell Physiol. 51:328-332].

As a technique for mass-producing a protein of interest in plants, Korean Patent Laid-Open Publication No. 10-2021-0117808 proposed a method for mass-producing a protein of interest by using a recombinant vector prepared by sequentially linking the MacT promoter which has a higher transcription level than the original Mac promoter by introducing a single nucleotide modification at the 3′ end of the Mac promoter, the M domain which increases protein expression, BiP which induces high accumulation in the ER, HDEL which induces a high accumulation rate in the ER, the termination site of RD29B, p38 which is a gene silencing suppressor, and the 5′-UTR which is a translation amplification sequence. However, there is no known technique for a method of recombining strong promoter sites derived from plant viruses to create a super promoter and repeatedly linking two types of terminators to produce a strong terminator, thereby ultimately increasing the expression of a gene of interest.

### DISCLOSURE

### Technical Problem

The present invention has been devised to solve the above-mentioned problems, and in order to achieve the high expression of a protein of interest, an object of the present invention is to provide a gene construct including a super promoter, produced by recombining strong promoter sites derived from plant viruses, and/or a strong terminator, produced by repeatedly connecting two types of terminators

Another object of the present invention is to provide a recombinant expression vector for mass-producing a protein of interest including the above-described gene construct, a transformant transformed with the recombinant expression vector, and a plant cell and a plant, into which the transformant is introduced.

Still another object of the present invention is to provide a method for producing a plant for mass-producing a protein of interest by using a transformant transformed with the above-described recombinant expression vector.

Still another object of the present invention is to provide a method for mass-producing a protein of interest in a plant by using a transformant transformed with the above-described recombinant expression vector.

### Technical Solution

In order to solve the above-described problems, the present invention provides a gene construct for high expression of a gene of a protein of interest, including an FMM-UD promoter in which the following (i) and (ii) are sequentially linked:

(i) an FMM promoter in which a Figwort subgenomic transcript promoter gene fragment, a Mirabilis mosaic virus subgenomic transcript promoter gene fragment and a Mirabilis mosaic virus full-length transcript promoter gene fragment are sequentially linked; and

(ii) an upstream DNA (UD) sequence with 4 repetitions of an upstream activation sequence (UAS), which is a GAL4-binding site of yeast, linked to a 5′ end of the FMM promoter.

According to another preferred embodiment of the present invention, the Figwort subgenomic transcript promoter gene may include a nucleotide sequence of SEQ ID NO: 1, the Mirabilis mosaic virus subgenomic transcript promoter gene may include a nucleotide sequence of SEQ ID NO: 2, and the Mirabilis mosaic virus full-length transcript promoter gene may include a nucleotide sequence of SEQ ID NO: 3.

According to still another embodiment of the present invention, the FMM-UD promoter gene may include a nucleotide sequence of SEQ ID NO: 4.

According to another preferred embodiment of the present invention, a TATA box sequence may be included at a 3′ end of the FMM-UD promoter, and a 5′ UTR gene may be linked to a 5′ end of the FMM-UD promoter.

According to still another preferred embodiment of the present invention, the 5′ UTR gene may include a nucleotide sequence of SEQ ID NO: 5.

In another aspect, the present invention provides a gene construct for high expression of a gene of a protein of interest, including a 3PR terminator in which a cauliflower mosaic virus 35S terminator gene, a 3′ region of a potato proteinase inhibitor II gene, and a gene of an RB7 scaffold attachment site are sequentially linked.

According to a preferred embodiment of the present invention, the cauliflower mosaic virus 35S terminator gene may include a nucleotide sequence of SEQ ID NO: 6, the 3′ region of the potato proteinase inhibitor II gene may include a nucleotide sequence of SEQ ID NO: 7, and the gene of the RB7 scaffold attachment site may include a nucleotide sequence of SEQ ID NO: 8.

According to another preferred embodiment of the present invention, the 3PR terminator may include a nucleotide sequence of SEQ ID NO: 9.

In still another aspect, the present invention provides a gene construct for high expression of a gene of a protein of interest, including the following (i) and (ii):

(i) an FMM-UD promoter including an FMM promoter in which a Figwort subgenomic transcript promoter gene fragment, a Mirabilis mosaic virus subgenomic transcript promoter gene fragment and a Mirabilis mosaic virus full-length transcript promoter gene fragment are sequentially linked, and an upstream DNA (UD) sequence with 4 repetitions of an upstream activation sequence (UAS), which is a GAL4-binding site of yeast, linked to a 5′ end of the FMM promoter; and

(ii) a 3PR terminator in which a cauliflower mosaic virus 35S terminator gene, a 3′ region of a potato proteinase inhibitor II gene, and a gene of an RB7 scaffold attachment site are sequentially linked.

According to a preferred embodiment of the present invention, the gene construct may further include a gene for encoding a protein of interest.

According to another preferred embodiment of the present invention, the protein of interest may be at least any one protein selected from the group consisting of human interleukin 6, a transcription factor, a toxic protein, a hormone, a hormone analog, a cytokine, a movement protein, an enzyme, an enzyme inhibitor, a transport protein, a structural protein, a receptor, a receptor fragment, a biological defense inducer, a storage protein, an exploitative protein, a reporter protein, an antigen, an antibody and an antibody fragment.

According to still another preferred embodiment of the present invention, the gene construct may include an expression cassette including a chaperone binding protein (BiP) gene, a mannosylated peptide region (MP) gene, a cellulose-binding module 3 (CBM3) gene, a small ubiquitin-related modifier (SUMO) gene, a gene encoding a protein of interest and a gene encoding His-Asp-Glu-Leu (HDEL) between (i) and (ii).

According to another preferred embodiment of the present invention, an amino acid sequence of the BiP may include an amino acid sequence of SEQ ID NO: 11, an amino acid sequence of the MP may include an amino acid sequence of SEQ ID NO: 13, an amino acid sequence of the CBM3 may include an amino acid sequence of SEQ ID NO: 17, and an amino acid sequence of the SUMO may include an amino acid sequence of SEQ ID NO: 19.

According to still another preferred embodiment of the present invention, the BiP gene may include a nucleotide sequence of SEQ ID NO: 10, the MP gene may include a nucleotide sequence of SEQ ID NO: 12, the CBM3 gene may include a nucleotide sequence of SEQ ID NO: 16, and the SUMO gene may include a nucleotide sequence of SEQ ID NO: 18.

In another aspect, the present invention provides a recombinant expression vector for high expression of a gene of a protein of interest, including various forms of the gene construct described above; and a gene encoding a protein of interest.

In addition, the present invention provides a transformant for mass-producing a protein of interest, transformed with the recombinant expression vector described above.

According to a preferred embodiment of the present invention, the transformant may be Agrobacterium.

Furthermore, the present invention provides a plant cell and a plant, into which the above-described transformant is introduced.

According to a preferred embodiment of the present invention, the plant may be selected from food crops including rice, wheat, barley, corn, soybeans, potatoes, wheat, red beans, oats and sorghum; vegetable crops including Arabidopsis, cabbage, radish, pepper, strawberry, tomato, watermelon, cucumber, cabbage, Korean melon, pumpkin, green onion, onion and carrot; specialty crops including ginseng, tobacco, cotton, sesame, sugarcane, sugar beet, perilla, peanut and rapeseed; fruit trees including apple trees, pear trees, jujube trees, peaches, grapes, tangerines, persimmons, plums, apricots, lemons and bananas; and floriculture including roses, carnations, chrysanthemums, lilies, sunflowers, cosmos and tulips.

Additionally, the present invention provides a method for producing a plant for mass-producing a protein of interest, the method including:
(a) constructing the above-described recombinant expression vector;
(b) producing a transformant transformed with the recombinant expression vector of step (a);
(c) culturing the transformant of step (b); and
(d) introducing a culture of the transformant into a plant.

According to a preferred embodiment of the present invention, the producing a transformant in step (b) may include any one method selected from the group consisting of an Agrobacterium sp.-mediated method, particle gun bombardment, sonication, electroporation and a polyethylene glycol (PEG)-mediated transformation method.

Additionally, the present invention provides a method for mass-producing a protein of interest, the method including:
(a) constructing the above-described recombinant expression vector;
(b) producing a transformant transformed with the recombinant expression vector of step (a);
(c) culturing the transformant of step (b);
(d) introducing a culture of the transformant into a plant; and
(e) pulverizing the plant of step (d) to extract a protein of interest.

According to another preferred embodiment of the present invention, the introducing a culture of the transformant into a plant in step (d) may include introducing a culture of the transformant into a leaf of a plant by syringe infiltration or vacuum infiltration.

### Advantageous Effects

The promoter and terminator according to the present invention have the effect of increasing the gene expression level of a protein of interest by being linked to a gene encoding the protein of interest to be produced. In detail, the FMM-UD promoter, which is a super promoter produced by recombining strong promoter sites derived from plant viruses, and/or the strong 3PR terminator produced by repeatedly linking two types of terminators improve the expression of a gene of a protein of interest, thereby enhancing the production of the protein of interest in a plant.

### DESCRIPTION OF DRAWINGS

FIG. 1 shows the results of comparing the efficiency of the FMM-UD promoter with the CaMV 35S promoter at the level of a protein of interest, and (A) is a concept map of the construct used in the experiment. The IL6 recombinant protein gene construct was constructed by using the material published in a previous study [Islam MR et al., (2019) Cost-effective production of tag-less recombinant protein in Nicotiana benthamiana. Plant Biotechnol J. 17:1094-1105]. The result on the left side of (B) shows that after transforming a plasmid construct in (A) into Agrobacterium, the Agrobacterium was temporarily infiltrated and expressed in Nicotiana benthamiana, and after extracting a protein, the same amount of the protein was reacted with an anti-hIL6 antibody to perform Western blot analysis. The result on the right side of (B) is the result of staining the Western blot membrane with Coomassie blue (M, standard protein size; NT, non-transgenic plant).

FIG. 2 shows the results of a comparative analysis of the efficiency of the FMM-UD promoter with the CaMV 35S promoter at the RNA level, where (A) is a conceptual map of the construct used in the experiment, and (B) is the result of transforming the plasmid construct of (A) into Agrobacterium, then temporarily infiltrating and expressing the Agrobacterium in Nicotiana benthamiana to extract total RNA, and performing quantitative reverse transcription-polymerase chain reaction (qRT-PCR) analysis.

FIG. 3 shows the results of a comparative analysis of the terminator effect for two types of promoters (CaMV 35S or FMM-UD) at the protein level, where (A) is a concept map of the construct used in the experiment, and (B) is a common vector used in the construct in (A). The result on the right side of (C) is the result of transforming the plasmid construct of (B) into Agrobacterium, and after the Agrobacterium was temporarily infiltrated and expressed in Nicotiana benthamiana for 3 days, the protein was extracted and Western blot analysis was performed by reacting with anti-hIL6 antibody, and the result on the left side of (C) is the result of staining the membrane on the right side of (C) with Coomassie blue. The result on the right side of (D) is the result of Western blot analysis performed by extracting proteins from the transformed plant in (C) 5 days later and reacting the same with an anti-hIL6 antibody. The result on the left side of (D) is the result of staining the membrane on the right side of (D) with Coomassie blue (NT, non-transformed control; M, standard protein size; RbcL, Rubisco large subunit; p38, gene silencing suppressor).

FIG. 4 shows the results of comparing and analyzing the effects of these combinations at the protein level by combining two types of promoters (CaMV 35S or FMM-UD) and two types of terminators (RD29BT or 3PRt), where (A) is a concept map of the construct used in the experiment, and (B) is an entire map of vectors used in the constructs of (A). The result on the right side of (C) is the result of transforming the plasmid construct of (B) into Agrobacterium, then temporarily infiltrating and expressing the Agrobacterium into Nicotiana benthamiana to be introduced, extracting proteins 3 days later, reacting the same amount of the protein with an anti-hIL6 antibody, and performing Western blot analysis, and the result on the left side of (C) is the result of staining the membrane on the right side of (C) with Coomassie blue. The result on the right side of (D) is the result of Western blot analysis performed by extracting proteins from the transformed plant in (C) 5 days later and reacting the same amount of the protein with an anti-hIL6 antibody, and the result on the left side of (D) is the result of staining the membrane on the right side of (C) with Coomassie blue (NT, non-transformed control; M, standard protein size; RbcL, Rubisco large subunit; p38, gene silencing suppressor).

FIG. 5 is a vector map showing the basic structure of a binary vector used in the present invention.

### BEST MODE

As described above, producing proteins in plants, unlike producing proteins in microorganisms or animal cells, is relatively safe from endotoxins and viruses that infect humans and animals, and has the advantage of being able to produce proteins in large quantities at low cost. Accordingly, in the present invention, in order to increase the gene expression level of a protein of interest in plants, a recombinant promoter and a recombination terminator were produced, and each or all of them were linked to a gene of a protein of interest to construct a recombinant expression vector for the high expression of the protein of interest. The recombinant expression vector constructed in the present invention was introduced into a host cell such as Agrobacterium to prepare a transformant, and the transformant was infiltrated into a plant such as Nicotiana benthamiana (N. benthamiana). As a result, it was confirmed that the expression level of the protein of interest increased in the plant into which the transformant was introduced.

Therefore, the present invention relates to a gene construct, including a recombinant promoter constructed for the high expression of a protein of interest.

Specifically, the recombinant promoter may include an FMM-UD promoter gene including an FMM promoter in which a Figwort subgenomic transcript promoter gene fragment, a Mirabilis mosaic virus subgenomic transcript promoter gene fragment and a Mirabilis mosaic virus full-length transcript promoter gene fragment are sequentially linked; and an upstream DNA (UD) sequence with 4 repetitions of an upstream activation sequence (UAS), which is a GAL4-binding site of yeast, linked to a 5′ end of the FMM promoter

According to another preferred embodiment of the present invention, the Figwort subgenomic transcript promoter gene may include a nucleotide sequence of SEQ ID NO: 1, the Mirabilis mosaic virus subgenomic transcript promoter gene may include a nucleotide sequence of SEQ ID NO: 2, and the Mirabilis mosaic virus full-length transcript promoter gene may include a nucleotide sequence of SEQ ID NO: 3.

In this case, between each viral promoter gene, the nucleotide sequence of a connection site for linking two viral promoter genes may be optionally included, that is, the nucleotide sequence of a connection site may or may not be included.

According to still another preferred embodiment of the present invention, the FMM-UD promoter gene may include a nucleotide sequence of SEQ ID NO: 4.

According to another preferred embodiment of the present invention, a TATA box sequence may be included at a 3′ end of the FMM-UD promoter, and a 5′ UTR gene may be linked to a 5′ end of the FMM-UD promoter as a translation amplification sequence.

According to still another preferred embodiment of the present invention, the 5′ UTR gene may include a nucleotide sequence of SEQ ID NO: 5.

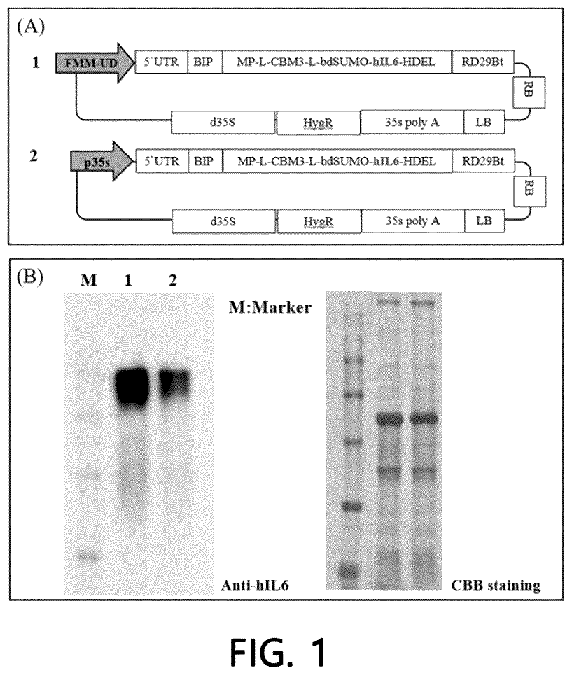

In a specific embodiment of the present invention, in order to develop a promoter that is stronger than the existing strong promoter, CaMV 35S promoter, the inventors of the present invention sequentially fused a Figwort subgenomic transcript promoter (nucleotide sequence at position −270 to −63 from the transcription start site, SEQ ID NO: 1), a Mirabilis mosaic virus subgenomic transcript promoter (nucleotide sequence at position −306 to −125 from the transcription start site, SEQ ID NO: 2), and a Mirabilis mosaic virus full-length transcript promoter (nucleotide sequence at position −193 to +63 from the transcription start site, SEQ ID NO: 3) derived from plant viruses, and further added a GAL4-binding gene sequence to the above fused promoter to finally construct an FMM-UD promoter including a nucleotide sequence of SEQ ID NO: 4. As a result of expressing a gene sequence (SEQ ID NO: 20) of the recombinant protein prepare to produce hIL6, which is a protein of interest, by respectively using the FMM-UD promoter produced in the present invention and the conventionally known CaMV35S promoter, as confirmed in FIG. 1, the protein expression of the FMM-UD promoter was about 4 times higher than that of the CaMV35S promoter.

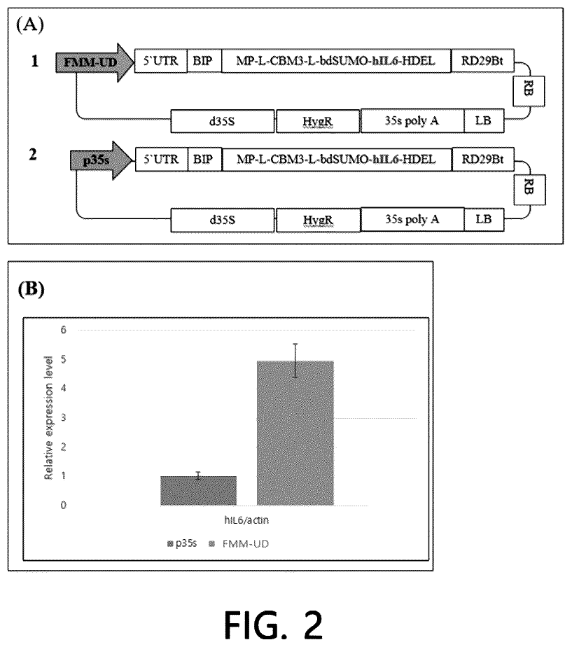

In another specific embodiment of the present invention, quantitative reverse transcription gene amplification technique (qRT-PCR) was used to explain an increase in the transcription level of a protein of interest by the promoter. As a result of comparing the transcriptional levels after expressing a gene sequence (SEQ ID NO: 20) of hIL6, which is the protein of interest, by respectively using the FMM-UD promoter and the CaMV 35S promoter, it was confirmed that as shown in FIG. 2, a transcription level approximately 5 times higher was observed in the FMM-UD promoter.

In another aspect, the present invention provides a gene construct for high expression of a protein of interest, including a 3PR terminator in which a cauliflower mosaic virus 35S terminator gene, a 3′ region of a potato proteinase inhibitor II gene, and a gene of an RB7 scaffold attachment site are sequentially linked.

According to a preferred embodiment of the present invention, the cauliflower mosaic virus 35S terminator gene may include a nucleotide sequence of SEQ ID NO: 6, the 3′ region of the potato proteinase inhibitor II gene may include a nucleotide sequence of SEQ ID NO: 7, and the gene of the RB7 scaffold attachment site may include a nucleotide sequence of SEQ ID NO: 8.

According to another preferred embodiment of the present invention, the 3PR terminator may include a nucleotide sequence of SEQ ID NO: 9.

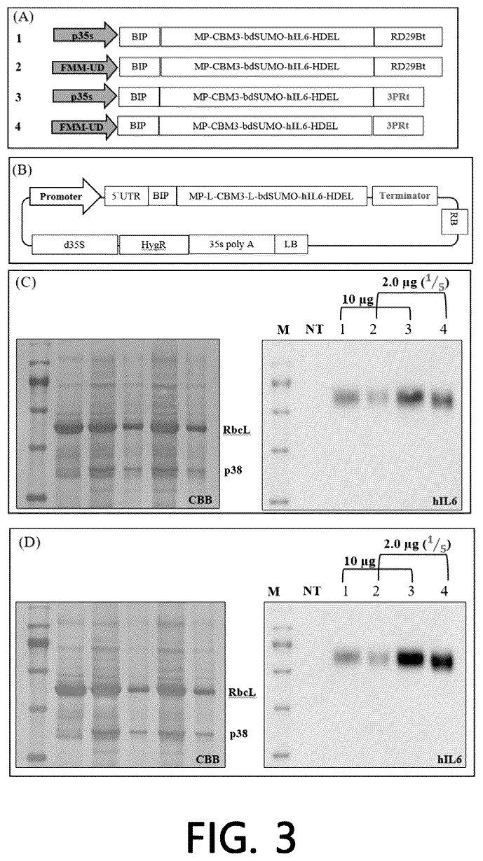

In a specific embodiment of the present invention, in order to develop a terminator that is stronger than the existing RD29Bt terminator, the inventors of the present invention constructed a 3PR terminator in which a cauliflower mosaic virus 35S terminator gene (SEQ ID NO: 6), a 3′ region of a potato proteinase inhibitor II gene (SEQ ID NO: 7), and a gene of an RB7 scaffold attachment site (SEQ ID NO: 8) are sequentially linked. In order to determine the expression level of a protein of interest according to the type of terminator, as shown in (A) of FIG. 3, the conventionally known RD29Bt terminator and the 3PR terminator prepared in the present invention were introduced into the CaMV 35S promoter and the FMM-UD promoter, respectively, and after these were infiltrated into Agrobacterium, these were infiltrated into Nicotiana benthamiana, and the expression level of hIL6, which is the protein of interest was determined after 3 and 5 days. As a result, as shown in (C) of FIG. 3, when the 3PR terminator was included for the same promoter, it was confirmed that the expression of the protein of interest was approximately 2 times higher after 3 days, and as shown in (D) of FIG. 3, it was confirmed that the expression was approximately 3 times higher after 5 days.

Accordingly, the present invention provides a gene construct for high expression of a protein of interest, including the following (i) and (ii):

(i) an FMM-UD promoter including an FMM promoter in which a Figwort subgenomic transcript promoter gene fragment, a Mirabilis mosaic virus subgenomic transcript promoter gene fragment and a Mirabilis mosaic virus full-length transcript promoter gene fragment are sequentially linked, and an upstream DNA (UD) sequence with 4 repetitions of an upstream activation sequence (UAS), which is a GAL4-binding site of yeast, linked to a 5′ end of the FMM promoter; and

(ii) a 3PR terminator in which a cauliflower mosaic virus 35S terminator gene, a 3′ region of a potato proteinase inhibitor II gene, and a gene of an RB7 scaffold attachment site are sequentially linked.

According to a preferred embodiment of the present invention, the gene construct may further include a gene encoding a protein of interest.

The “protein of interest” is a term meaning a protein to be produced, and it may be any type of protein that can be expressed as a recombinant protein. The gene construct includes genes encoding intracellular and foreign proteins to be expressed. For example, a protein of interest may be any one or more selected from the group consisting of an interleukin, transcription factor, membrane protein, insulin, cytokinin, growth factor, toxin protein, hormone, hormone analog, cytokine, movement protein, lysozyme, vaccine, enzyme, enzyme inhibitor, transport protein, structural protein, receptor, receptor fragment, biodefense inducer, storage protein, exploitive protein, reporter protein, antigen, antibody and antibody fragment. The gene encoding this protein of interest may include a “cloning site”, which is a nucleic acid sequence into which a restriction enzyme recognition or cleavage site is introduced, such that it can be inserted into a vector.

According to a preferred embodiment of the present invention, the interleukin includes human interleukin 6, but is not limited thereto. The human-derived interleukin 6 gene may include a nucleotide sequence of SEQ ID NO: 20, and the human-derived interleukin 6 protein may include an amino acid sequence of SEQ ID NO: 21.

According to still another preferred embodiment of the present invention, the gene construct may include an expression cassette including a chaperone binding protein (BiP) gene, a mannosylated peptide region (MP) gene, a cellulose-binding module 3 (CBM3) gene, a small ubiquitin-related modifier (SUMO) gene, a gene encoding a protein of interest and a gene encoding His-Asp-Glu-Leu (HDEL) between (i) and (ii).

In this case, the SUMO gene may be derived from Brachypodium distachyon, and the CBM3 gene may be derived from Clostridium thermocellum, but are not limited thereto.

According to another preferred embodiment of the present invention, a suitable linker, for example, a peptide linker of 1 to 50 amino acids in length, 1 to 30 amino acids in length, 1 to 20 amino acids in length, 2 to 50 amino acids in length, 2 to 30 amino acids in length or 2 to 20 amino acids in length may be included between the mannosylated peptide region (MP) gene and the cellulose-binding module 3 (CBM3) gene and between the cellulose-binding module 3 (CBM3) gene and the small ubiquitin-related modifier (SUMO) gene. The peptide linker may be a repeating glycine-serine sequence, but is not limited thereto.

According to a preferred embodiment of the present invention, the gene sequence of the linker may include a nucleotide sequence of SEQ ID NO: 14, and the protein sequence of the linker may include an amino acid sequence of SEQ ID NO: 15.

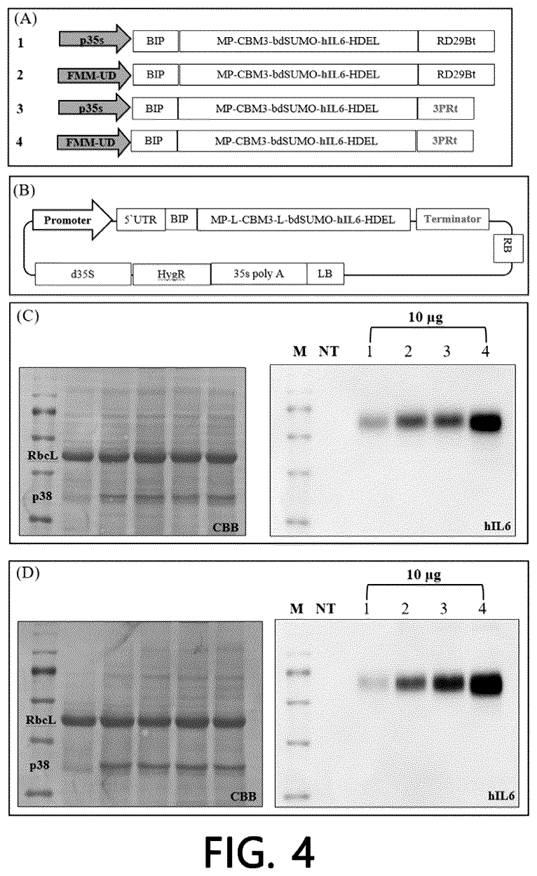

In a specific embodiment of the present invention, the inventors of the present invention confirmed that based on the results of FIGS. 1 to 3, the FMM-UD promoter and the 3PR terminator can independently enhance the protein level of a protein of interest, and thus, an expression cassette was constructed by inserting BiP-MP-CBM3-bdSUMO-hIL6-HDEL (SEQ ID NO: 20) between the above [Islam MR et al., (2019) Cost-effective production of tag-less recombinant protein in Nicotiana benthamiana. Plant Biotechnol J. 17:1094-1105], and the expression level of a protein of interest according to the combination of the FMM-UD promoter and the 3PR terminator was further determined. Chaperone binding protein (BiP) is for targeting recombinant proteins including hIL6, which is a protein of interest, to the endoplasmic reticulum, and mannosylated peptide region (MP) is a fragment consisting of 60 amino acid residues from alanine at position 231 to aspartic acid at position 290 of protein tyrosine phosphatase, receptor type, C (PTPRC), and these are for increasing the level of protein expression. CBM3 is a portion that can bind to microcrystalline (MCC) beads for subsequent protein purification, the bdSUMO gene increases the solubility of a protein, hIL6 is a human interleukin and corresponds to the protein of interset in the present invention, and HDEL is for keeping the protein in the endoplasmic reticulum. As shown in (A) of FIG. 4, 35S: : RD29B-t was constructed as a reference construct, and BiP-MP-CBM3-bdSUMO-hIL6-HDEL was used as a reporter gene to complete the expression construct. In order to compare the reference construct and FMM-UD::3PRt, after expressing the recombinant gene of hIL6 in Nicotiana benthamiana (N. benthamiana), the protein level was determined, and as a result, it was confirmed that as shown in (C) and (D) of FIG. 4, FMM-UD::3PRt showed the highest protein level. Therefore, the combination of FMM-UD and 3PRt was established as a combination of a novel promoter and a terminator for high expression of a protein of interest in plants. By linking a BiP signal sequence to the N-terminus of the gene for a recombinant protein and linking an HDEL signal sequence to the C-terminus, the effect of accumulating the protein of interest at a high concentration in the endoplasmic reticulum (ER) may be achieved.

However, the nucleotide sequence that can target a protein of interest to the endoplasmic reticulum is not limited to BiP, and additionally, various signal peptides involved in targeting to the endoplasmic reticulum may be used without limitation. The nucleotide sequence capable of maintaining the protein of interest in the endoplasmic reticulum is not limited thereto, but preferably, a nucleotide sequence encoding a peptide that can be selected from a combination of [His/Lys/Arg][Asp/Glu]Glu-Leu may be used. Since the N-terminus of the BiP protein contains a signal peptide that determines targeting to the endoplasmic reticulum, it may play a role in targeting proteins of interest to the endoplasmic reticulum. The signal peptide inserted for targeting to the endoplasmic reticulum is not limited to the signal peptide at the N-terminus of the BiP protein, and various signal peptides involved in targeting to the endoplasmic reticulum may be used.

According to another preferred embodiment of the present invention, an amino acid sequence of the BiP may include an amino acid sequence of SEQ ID NO: 11, an amino acid sequence of the MP may include an amino acid sequence of SEQ ID NO: 13, an amino acid sequence of the CBM3 may include an amino acid sequence of SEQ ID NO: 17, and an amino acid sequence of the SUMO may include an amino acid sequence of SEQ ID NO: 19.

According to still another preferred embodiment of the present invention, the BiP gene may include a nucleotide sequence of SEQ ID NO: 10, the MP gene may include a nucleotide sequence of SEQ ID NO: 12, the CBM3 gene may include a nucleotide sequence of SEQ ID NO: 16, and the SUMO gene may include a nucleotide sequence of SEQ ID NO: 18.

The amino acid sequences and nucleic acid sequences described herein may be interpreted by expanding to sequences having at least 70%, at least 75%, at least 80%, at least 85%, at least 90%, at least 91%, at least 92%, at least 93%, at least 94%, at least 95%, at least 96%, at least 97%, at least 98%, or at least 99% homology.

The “% of sequence homology” may be determined by comparing a comparison region with the two optimally aligned sequences, and a portion of the nucleotide sequence in the comparison region may include additions or deletions (i.e. gaps) compared to a reference sequence (not including additions or deletions) for the optimal alignment of the two sequences.

In another aspect, the present invention provides a recombinant expression vector for mass-producing a protein of interest in a plant, including various forms of the gene construct described above; and a gene encoding a protein of interest.

In the recombinant expression vector, the gene construct and a gene encoding a protein of interest are operably linked.

In the present invention, the “recombinant expression vector” refers to a plasmid, virus or other mediator known in the art, into which various types of the gene constructs described above may be inserted or introduced. Various types of the gene constructs according to the present invention may be operably linked, and the operably linked gene constructs may be included in one expression vector including both of a selection marker and a replication origin. The term “operably linked” may be a gene and an expression control sequence that are linked in a manner that allows gene expression, when an appropriate molecule is linked to the expression control sequence. “An expression control sequence” refers to a DNA sequence that regulates the expression of an operably linked nucleotide sequence in a specific host cell. These control sequences include a promoter to implement transcription, optional operator sequences to regulate transcription, sequences encoding suitable mRNA ribosome binding sites, and sequences that regulate the termination of transcription and translation.

The “recombinant expression vector” may be at least one selected from the group consisting of all plasmids, phages, yeast plasmids, plant cell viruses, mammalian cell viruses and other carriers known in the art, into which a genetic sequence or nucleotide sequence may be inserted or introduced. In general, any plasmid and vector may be used without particular limitation as long as it can replicate and stabilize within a plant cell or plant host. Suitable vectors for introducing various types of the gene constructs described above in the present invention include Ti plasmids and plant virus vectors. Examples of known vectors include pBI121, pHellsgate8, pROKII, pBI76, pET21, pSK(+), pLSAGPT, pUC and pGEM. In addition, for vectors expressed in a plant, including the CMV35s promoter, for example, the pCAMBIA series (pCAMBIA1200, 1201, 1281, 1291, 1300, 1301, 1302, 1303, 1304, 1380, 1381, 2200, 2201, 2300, 2301, 3200, 3201, 3300), pMDC32 and pC-TAPapYL436 may be used, but are not limited thereto. A person skilled in the art may select a vector suitable for introducing the gene construct of the present invention, and in the present invention, any vector that can introduce various types of the gene constructs described above into a plant cell may be used.

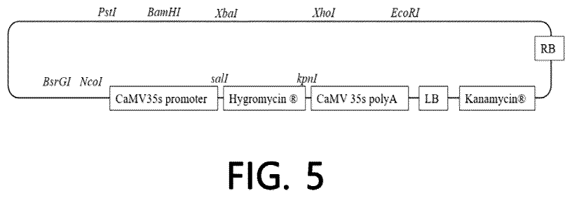

In a specific embodiment of the present invention, a recombinant vector was prepared by inserting an FMM-UD promoter-protein of interest-3PR terminator into a vector of FIG. 5 by using the restriction enzyme PstI and EcoRI sites.

In addition, the present invention provides a transformant for mass-producing a protein of interest, transformed with the above-described recombinant expression vector.

According to a preferred embodiment of the present invention, the transformant may be Agrobacterium.

Furthermore, the present invention provides a plant cell and a plant, into which the above-described transformant has been introduced.

According to a preferred embodiment of the present invention, the plant may be selected from food crops including rice, wheat, barley, corn, soybeans, potatoes, wheat, red beans, oats and sorghum; vegetable crops including Arabidopsis, cabbage, radish, pepper, strawberry, tomato, watermelon, cucumber, cabbage, Korean melon, pumpkin, green onion, onion and carrot; specialty crops including ginseng, tobacco, cotton, sesame, sugarcane, sugar beet, perilla, peanut and rapeseed; fruit trees including apple trees, pear trees, jujube trees, peaches, grapes, tangerines, persimmons, plums, apricots, lemons and bananas; and floriculture including roses, carnations, chrysanthemums, lilies, sunflowers, cosmos and tulips.

Additionally, the present invention provides a method for producing a plant for mass-producing a protein of interest, the method including:
(a) constructing the above-described recombinant expression vector;
(b) producing a transformant transformed with the recombinant expression vector of step (a);
(c) culturing the transformant of step (b); and
(d) introducing a culture of the transformant into a plant.

According to a preferred embodiment of the present invention, the producing a transformant in step (b) may include any one method selected from the group consisting of an Agrobacterium sp.-mediated method, particle gun bombardment, sonication, electroporation and a polyethylene glycol (PEG)-mediated transformation method.

Additionally, the present invention provides a method for mass-producing a protein of interest, the method including:
(a) constructing the above-described recombinant expression vector;
(b) producing a transformant transformed with the recombinant expression vector of step (a);
(c) culturing the transformant of step (b);
(d) introducing a culture of the transformant into a plant; and
(e) pulverizing the plant of step (d) to extract a protein of interest.

In the method for mass-producing a protein of interest according to the present invention, the method of introducing a culture of the transformant into a plant in step (d) may include introducing a culture of the transformant into a leaf of a plant by syringe infiltration or vacuum infiltration. The Agrobacterium injected in this way receives a signal from an acetosyringone substance and delivers a promoter-protein of interest-terminator construct of the vector into a plant cell.

In the method for mass-producing a protein of interest according to the present invention, extracting a protein of interest in step (e) may be performed through various separation and purification methods known in the art, and conventionally, in order to remove cell debris and the like, the cell lysate may be centrifuged, and then, precipitation, for example, salting out (ammonium sulfate precipitation and sodium phosphate precipitation), solvent precipitation (protein fraction precipitation using acetone, ethanol, etc.) and the like may be performed, and dialysis, electrophoresis, and various types of column chromatography and the like may be performed. For the chromatography, the protein of interest of the present invention may be purified by applying techniques such as ion exchange chromatography, gel-permeation chromatography, HPLC, reverse phase-HPLC, affinity column chromatography or ultrafiltration, alone or in combination.

Hereinafter, the present invention will be described in more detail through examples. However, since the present invention can make various changes and take various forms, the specific embodiments and descriptions described below are only intended to aid understanding of the present invention and do not limit the present invention to the specific disclosed form. The scope of the present invention should be understood to include all changes, equivalents and substitutes included in the spirit and technical scope of the present invention.

### MODES OF THE INVENTION

### Example 1

### Confirmation of the Effect of Promoter Showing Higher Expression Level Than CaMV 35S at the Level of Protein of Interest

As shown in (A) of FIG. 1, an expression vector was constructed, and FMM-UD or CaMV 35S was used as a promoter. A binary vector for expression was completed by linking human interleukin 6 (hIL6), which is a soluble protein, to each of the two types of promoters with an RD29B terminator. The vector shown in FIG. 5 was used as the basic structure (backbone) of the binary vector. Each promoter-hIL6-RD29Bt construct was introduced into a binary vector using pstI and EcoI restriction enzyme sites. The binary vector was transformed into Agrobacterium by electric shock, and the transformed Agrobacterium was cultured in LB medium including the antibiotics kanamycin and rifampicin (50 mg/L and 100 mg/L, respectively) in a light-free environment at 28° C. for 40 hours. One colony was selected and cultured in LB medium including 5 mL of kanamycin and rifampicin (50 mg/L and 100 mg/L, respectively) for 15 hours in the dark with shaking. After 15 hours, the Agrobacterium was incubated with an infiltration buffer (10 mM MES, 10 mM MgSO₄) and 200 μM of acetosyringone, which is a plant signaling substance, at room temperature for 1 hour and 30 minutes without shaking.

The Agrobacterium was injected into a Nicotiana benthamiana (N. benthamiana) plant 5 weeks after germination by using a 1 mL syringe. After 3 days from the plant, leaves were harvested, nitrogen-fixed and pulverized to extract proteins. The protein extract was extracted by using an extraction solution [50 mM Tris pH 7.5, 150 mM NaCl, 1 mM EDTA, 1% Triton X100, and 1% protease inhibitor cocktail], and the supernatant of the corresponding extract was added to a final concentration of 1Y with a 6Y sample solution [500 mM Tris-HCl (pH 6.8), 10% SDS, 0.5% bromophenol blue, 30% glycerol (v/v), and 100 mM DTT] to prepare an electrophoresis protein sample. The sample extracted through the above process was boiled for 5 minutes, separated through 10% SDS-PAGE, and then subjected to western blot analysis using anti-hIL6 antibody. Through this, the expression levels of each promoter of CaMV 35S and FMM-UD were compared and analyzed. As a result, as shown in (B) of FIG. 1, it was confirmed that the FMM-UD promoter was significantly higher than CaMV 35S, and was a promoter that was at least 4 times stronger than the CaMV 35S promoter.

### Example 2

### Confirmation of the Effect of Promoter Showing Higher Expression Level Than CaMV 35S at the Level of RNA of Interest

In the same manner as Example 1, the CaMV 35S and FMM-UD promoters were linked to the RD29B terminator and the protein of interest, hIL6, respectively, and introduced into a binary vector to construct an expression vector as shown in (A) of FIG. 2. The binary vector was transformed into Agrobacterium, the Agrobacterium was cultured by the method described in Example 1, and then infiltrated into a plant. The leaves of the infiltrated plant were harvested 3 days later, RNA was extracted using the RNeasy kit (QIAGEN), and cDNA was synthesized by performing PCR using the extracted RNA and hIL6 primer. Quantitative reverse transcription gene amplification (qRT-PCR) was performed on the cDNA using a qRT-PCR kit (Invitrogen) to determine the activities of the CaMV 35S promoter and FMM-UD promoter at the RNA level. Actin was used as a reference gene for relative quantification. As a result, as shown in (B) of FIG. 2, it was confirmed that the FMM-UD promoter is a promoter about 5 times stronger at the transcription level than the CaMV 35S promoter.

### Example 3

### Identification of Termination Sites Showing Optimal Expression Levels

Expression constructs were constructed as shown in (A) of FIG. 3. The CaMV 35S or FMM-UD promoter was used as a promoter, and the RD29B terminator, which is known to be highly efficient, was used as a termination site as a control group, and the 3PR terminator was used as an experimental group. An expression cassette was constructed by inserting a BiP-CBM3-bdSUMO-hIL6-HDEL gene, which includes BiP which allows proteins to remain in the endoplasmic reticulum, CBM3 which is required for subsequent protein purification, bdSUMO which increases protein solubility, human interleukin 6 (hIL6) which is a protein of interest, and HDEL which is a signal for transporting to the endoplasmic reticulum.

The expression cassette designed as above was introduced into a binary vector using restriction enzymes, as shown in (B) of FIG. 3. The restriction enzyme sites used are PstI and EcoRI. Each of these constructs was transformed into Agrobacterium by electric shock, and then cultured in LB medium including kanamycin and rifampicin (50 mg/L and 100 mg/L, respectively) in a light-free environment at 28° C. for 40 hours. A single colony was cultured in 5 mL of LB medium including kanamycin and rifampicin with shaking in a dark environment for 15 hours. After 15 hours, the Agrobacterium was treated with an infiltration buffer (10 mM MES, 10 mM MgSO₄) and 200 μM of acetosyringone, which is a plant signaling substance, at room temperature for 1 hour and 30 minutes without shaking.

The Agrobacterium solution was injected into the entire leaves of a Nicotiana benthamiana (N. benthamiana) plant 5 weeks after germination using a 1 mL syringe. Leaves were harvested from the Agrobacterium-injected plant 3 and 5 days later, and protein was extracted. Protein extracts were extracted by using an extraction solution (50 mM Tris pH 7.5, 150 mM NaCl, 1 mM EDTA, 1% Triton X100, and 1% protease inhibitor cocktail), and a 6Y sample solution [500 mM Tris-HCl (pH 6.8), 10% SDS, 0.5% bromophenol blue, 30% glycerol (v/v), and 100 mM DTT] was added herein to a final 1Y concentration to prepare an electrophoresis protein sample. The sample extracted through the above process was boiled for 5 minutes, separated through 10% SDS-PAGE, and then subjected to western blot analysis using anti-hIL6 antibody. Afterwards, the membrane was stained with Coomassie blue for 20 minutes. As a result, as shown in (C) and (D) of FIG. 3, it was confirmed that the 3PR terminator had a higher expression level than the RD29B terminator in both of the CaMV35s promoter and the FMM-UD promoter.

### Example 4

### Identification of the Optimal Combination of Promoter and Terminator Showing the Highest Expression

As shown in (A) of FIG. 4, four types of expression constructs, 35S::RD29Bt, FMM-UD::RD29Bt, 35S::3PRt and FMM-UD::3PRt, were constructed by using various combinations of promoters and termination sites. An expression cassette was created by inserting a BiP-CBM3-bdSUMO-hIL6-HDEL gene therebetween. The expression cassette designed as above was introduced into a binary vector as shown in (B) of FIG. 3 through restriction enzymes. The restriction enzymes used are PstI and EcoRI. Each of these constructs was transformed into Agrobacterium by electric shock, and then cultured in LB medium including kanamycin and rifampicin (50 mg/L and 100 mg/L, respectively) in a light-free environment at 28° C. for 40 hours. A single colony was cultured in 5 mL of LB medium including kanamycin and rifampicin in a dark environment with shaking for 15 hours. After 15 hours, the Agrobacterium was treated with an infiltration buffer (10 mM MES, 10 mM MgSO₄) and 200 μM of acetosyringone, which is a plant signaling substance, at room temperature for 1 hour and 30 minutes without shaking.

The Agrobacterium solution was injected into the entire leaves of a Nicotiana benthamiana (N. benthamiana) plant 5 weeks after germination by using a 1 mL syringe. Leaves were harvested from the Agrobacterium-injected plant 3 and 5 days later, and protein was extracted. Harvested leaves were immediately nitrogen-fixed, and the nitrogen-fixed leaves were pulverized by using an extraction solution (50 mM Tris pH 7.5, 150 mM NaCl, 1 mM EDTA, 1% Triton X100, and 1% protease inhibitor cocktail) to extract proteins. A 6Y sample solution [500 mM Tris-HCl (pH 6.8), 10% SDS, 0.5% bromophenol blue, 30% glycerol (v/v), and 100 mM DTT] was added herein to a final 1Y concentration to prepare an electrophoresis protein sample. The sample extracted through the above process was boiled for 5 minutes, separated through 10% SDS-PAGE, and then, western blot was performed by using an anti-hIL6 antibody. Afterwards, the membrane was then stained with Coomassie blue. As a result, as shown in (C) and (D) of FIG. 4, it was confirmed that the expression level was the highest when hIL6 was expressed by combining the FMM-UD promoter and the 3PR terminator.

The gene sequences and amino acid sequence information thereof used in the examples of the present invention are shown in Table 1 below.

TABLE 1

| | | SEQ |
|---|---|---|
| | | ID |
| Name | Nucleotide Sequence/Amino Acid Sequence | NO: |
| Figwort subgenomic | tttacagtaagaactgataacaaaaattttacttatttccttagaattaatcttaaag | 1 |
| transcript promoter | gtgatagtaaacaaggacgattagtccgttggcaaaattggttcagcaagtatc | |
| (position −270 to −63 | aatttgatgtcgaacatcttgaaggtgtaaaaaacgttttagcagattgcctcac | |
| from transcription start | gagagattttaatgcttaaaaacgtaagcgctgacgtatga | |
| site) | | |
| Mirabilis mosaic virus | gttttacagtcaggacagataatgtaaatcttttaaaaggatttatgaataaaaag | 2 |
| subgenomic transcript | attactggtgacagtaaacagggaaggctaataagatggcaaatgtggttttca | |
| promoter (position −306 to | cattacacctttaaggtggaccacctaaaaggagaacaaaatgtgctggctgat | |
| −125 from transcription | tatctcaccagagaatt | |
| start site) | | |
| Mirabilis mosaic virus | aaaagatgatgcccgacagccacttgtgtgaagcatgtgaagccggtccctcc | 3 |
| full-length transcript | actaagaaaattagtgaagcatcttccagtggtccctccactcacagctcaatc | |
| promoter (position −193 | agtgagcaacaggacgaaggaaatgacgtaagccatgacgtctaatcccaca | |
| to +63 from transcription | agaatttccttatataaggaacacaaatcagaaggaagagatcaatcgaaatca | |
| start site) | aaatcggaatcgaaatcaaaatcggaatcgaaatctctcatct | |
| FMM-UD promoter | atccacgggtgacagccctccgacgggtgacagccctccgacgggtgacag | 4 |
| | ccctccgacgggtgacagccctccggattttacagtaagaactgataacaaaa | |
| | attttacttatttccttagaattaatcttaaaggtgatagtaaacaaggacgattagt | |
| | ccgttggcaaaattggttcagcaagtatcaatttgatgtcgaacatcttgaaggt | |
| | gtaaaaaacgttttagcagattgcctcacgagagattttaatgcttaaaaacgta | |
| | agcgctgacgtatgagttttacagtcaggacagataatgtaaatcttttaaaagg | |
| | atttatgaataaaaagattactggtgacagtaaacagggaaggctaataagatg | |
| | gcaaatgtggttttcacattacacctttaaggtggaccacctaaaaggagaaca | |
| | aaatgtgctggctgattatctcaccagagaattagcgtgggcggcgtgggcgt | |
| | agatctagcgtgggcggcgtgggcgtaaaagatgatgcccgacagccacttg | |
| | tgtgaagcatgtgaagccggtccctccactaagaaaattagtgaagcatcttcc | |
| | agtggtccctccactcacagctcaatcagtgagcaacaggacgaaggaaatg | |
| | acgtaagccatgacgtctaatcccacaagaatttccttatataaggaacacaaat | |
| | cagaaggaagagatcaatcgaaatcaaaatcggaatcgaaatcaaaatogga | |
| | atcgaaatctctcatct | |
| 5′ UTR | attattacatcaaaacaaaaa | 5 |
| CaMV 35S terminator | gtccgcaaaaatcaccagtctctctctacaaatctatctctctctatttttctccaga | 6 |
| | ataatgtgtgagtagttcccagataagggaattagggttcttatagggtttcgctc | |
| | atgtgttgagcatataagaaacccttagtatgtatttgtatttgtaaaatacttctat | |
| | caataaaatttctaattcctaaaaccaaaatccagtgac | |
| PI-II terminator | ctagagtcaccctgcaatgtgaccctagacttgtccatcttctggattggccaac | 7 |
| | ttaattaatgtatgaaataaaaggatgcacacatagtgacatgctaatcactataa | |
| | tgtgggcatcaaagttgtgtgttatgtgtaattactaattatctgaataagagaaa | |
| | gagatcatccatatttcttatcctaaatgaatgtcacgtgtctttataattctttgatg | |
| | aaccagatgcattttattaaccaattccatatacatataaatattaatcatatataatt | |
| | aatatcaattgggttagcaaaacaaatctagtctaggtgtgttttgctaattattgg | |
| | gggatagtgcaaaaagaaatctacgttctcaataattcagatagaaaacttaata | |
| | aagtgagataatttacatagattgcttttatcctttgatatatgtgaaaccatgcatg | |
| | atataaggaaaatagatagagaaataattttttacatcgttgaatatgtaaacaatt | |
| | taattcaagaagctaggaatataaatattgaggagtttatgatt | |
| RB7 | accaactoggtccatttgcacccctaatcataatagctttaatatttcaagatatta | 8 |
| | ttaagttaacgttgtcaatatcctggaaattttgcaaaatgaatcaagcctatatg | |
| | gctgtaatatgaatttaaaagcagctcgatgtggtggtaatatgtaatttacttgat | |
| | tctaaaaaaatatcccaagtattaataatttctgctaggaagaaggttagctacg | |
| | atttacagcaaagccagaatacaaagaaccataaagtgattgaagctcgaaat | |
| | atacgaaggaacaaatatttttaaaaaaatacgcaatgacttggaacaaaagaa | |
| | agtgatatattttttgttcttaaacaagcatcccctctaaagaatggcagttttccttt | |
| | gcatgtaactattatgctcccttcgttacaaaaattttggactactattgggaactt | |
| | cttctgaaaatagt | |
| 3PR terminator | gtccgcaaaaatcaccagtctctctctacaaatctatctctctctatttttctccaga | 9 |
| | ataatgtgtgagtagttcccagataagggaattagggttcttatagggtttcgctc | |
| | atgtgttgagcatataagaaacccttagtatgtatttgtatttgtaaaatacttctat | |
| | caataaaatttctaattcctaaaaccaaaatccagtgacgagctcctagagtcac | |
| | cctgcaatgtgaccctagacttgtccatcttctggattggccaacttaattaatgt | |
| | atgaaataaaaggatgcacacatagtgacatgctaatcactataatgtgggcat | |
| | caaagttgtgtgttatgtgtaattactaattatctgaataagagaaagagatcatc | |
| | catatttcttatcctaaatgaatgtcacgtgtctttataattctttgatgaaccagatg | |
| | cattttattaaccaattccatatacatataaatattaatcatatataattaatatcaatt | |
| | gggttagcaaaacaaatctagtctaggtgtgttttgctaattattgggggatagtg | |
| | caaaaagaaatctacgttctcaataattcagatagaaaacttaataaagtgagat | |
| | aatttacatagattgcttttatcctttgatatatgtgaaaccatgcatgatataagga | |
| | aaatagatagagaaataattttttacatcgttgaatatgtaaacaatttaattcaag | |
| | aagctaggaatataaatattgaggagtttatgattgagctcaccaacteggtcca | |
| | tttgcacccctaatcataatagctttaatatttcaagatattattaagttaacgttgtc | |
| | aatatcctggaaattttgcaaaatgaatcaagcctatatggctgtaatatgaattta | |
| | aaagcagctcgatgtggtggtaatatgtaatttacttgattctaaaaaaatatccc | |
| | aagtattaataatttctgctaggaagaaggttagctacgatttacagcaaagcca | |
| | gaatacaaagaaccataaagtgattgaagctcgaaatatacgaaggaacaaat | |
| | atttttaaaaaaatacgcaatgacttggaacaaaagaaagtgatatattttttgttc | |
| | ttaaacaagcatcccctctaaagaatggcagttttcctttgcatgtaactattatgc | |
| | tcccttcgttacaaaaattttggactactattgggaacttcttctgaaaatagtg | |
| BiP | atggctcgctcgtttggagctaacagtaccgttgtgttggcgatcatcttcttcgg | 10 |
| | tgagtgattttccgatcttcttctccgatttagatctcctctacattgttgcttaatctc | |
| | agaaccttttttcgttgttcctggatctgaatgtgtttgtttgcaatttcacgatctta | |
| | aaaggttagatctcgattggtattgacgattggaatctttacgatttcaggatgttt | |
| | atttgcgttgtcctctgcaatagaagaggctacgaagtta | |
| | MARSFGANSTVVLAIIFFGCLFALSSAIEEATKL | 11 |
| MP | gcaaacatcactgtggattacttatataacaaggaaactaaattatttacagcaa | 12 |
| | agctaaatgttaatgagaatgtggaatgtggaaacaatacttgcacaaacaatg | |
| | aggtgcataaccttacagaatgtaaaaatgcgtctgtttccatatctcataattcat | |
| | gtactgctcctgat | |
| | ANITVDYLYNKETKLFTAKLNVNENVECGNNTCT | 13 |
| | NNEVHNLTECKNASVSISHNSCTAPD | |
| Linker | ggtggaggtgggtctggtggtggatca | 14 |
| | GGGGSGGGS | 15 |
| CBM3 | gtatcaggtaaccttaaggtggagttttacaactcgaacccttctgatacaacta | 16 |
| | actcaataaacccacagttcaaagttacaaacacaggcagctctgcgatcgatt | |
| | tgtctaaattaaccctcagatactattatacggttgatggacagaaggaccaga | |
| | ctttctggtgtgatcatgcagctatcattggttctaacggtagctacaacggaatt | |
| | acatcaaacgtgaagggcactttcgttaagatgtcctctagcactaacaacgca | |
| | gacacatatttggagatcagttttacggggggaacccttgaaccaggtgctcac | |
| | gtccagattcaaggaagattcgctaaaaacgactggtcgaactatacccaaag | |
| | taatgattacagttttaaatccgcctcacaatttgttgagtgggatcaggtcactg | |
| | cttacctgaacggggttctagtgtggggaaaggaacctggtccc | |
| | VSGNLKVEFYNSNPSDTTNSINPQFKVTNTGSSAI | 17 |
| | DLSKLTLRYYYTVDGQKDQTFWCDHAAIIGSNGS | |
| | YNGITSNVKGTFVKMSSSTNNADTYLEISFTGGTL | |
| | EPGAHVQIQGRFAKNDWSNYTQSNDYSFKSASQF | |
| | VEWDQVTAYLNGVLVWGKEPGP | |
| bdSUMO | atgcatattaatttgaaagtgaagggacaggacggaaacgaggttttcttccgt | 18 |
| | atcaaaagatcaacacaactcaagaaactcatgaatgcttactgcgatagaca | |
| | aagcgtggacatgacagctatagctttcctatttgatgggagaaggttgagagc | |
| | ggaacagactccggatgagcttgaaatggaagatggagatgagattgacgca | |
| | atgttacatcagactggtggt | |
| | MHINLKVKGQDGNEVFFRIKRSTQLKKLMNAYC | 19 |
| | DRQSVDMTAIAFLFDGRRLRAEQTPDELEMEDGD | |
| | EIDAMLHQTGG | |
| hIL6 | atggttcctccaggagaggattctaaggatgtggctgcacctcatagacagcc | 20 |
| | attgacctcttcagaaagaatcgataagcaaattaggtacattctcgatggtatat | |
| | ctgctttaaggaaggagacatgtaataagtcaaacatgtgcgaaagttctaagg | |
| | aggctctcgcagaaaataaccttaatttgcctaagatggctgaaaaagatggat | |
| | gttttcagtctggtttcaacgaagagacttgccttgttaaaattatcacaggacttt | |
| | tggaatttgaggtgtatcttgaatacttacaaaacagattcgaatcaagtgaaga | |
| | gcaggctagggcagttcaaatgagtactaaggtgcttatacagttcttgcaaaa | |
| | gaaagctaagaaccttgatgcaatcactacacctgatccaaccactaatgettc | |
| | actcttaacaaagcttcaagcacagaaccaatggttgcaggatatgacaaccc | |
| | accttattctcaggtcattcaaggagtttttacagtcaagtcttagggctcttaggc | |
| | agatg | |
| | MVPPGEDSKDVAAPHRQPLTSSERIDKQIRYILDG | 21 |
| | ISALRKETCNKSNMCESSKEALAENNLNLPKMAE | |
| | KDGCFQSGFNEETCLVKIITGLLEFEVYLEYLQNR | |
| | FESSEEQARAVQMSTKVLIQFLQKKAKNLDAITTP | |
| | DPTTNASLLTKLQAQNQWLQDMTTHLILRSFKEF | |
| | LQSSLRALRQM | |
| HDEL | cacgatgagctc | 22 |
| | HDEL | 23 |

*The underlined part in the nucleotide sequence of SEQ ID NO: 10 represents an intron.

The national research and development projects that supported the present invention are as follows.

(1) [Project Identification Number] 1395067658

[Project Number] PJ015701012021

[Name of Ministry] Rural Development Administration

[Name of Project Management (Specialized) Organization] Rural Development Administration

[Research Project Name] Biogreen-linked agricultural life innovation technology development

[Research Task Name] Development of foot-and-mouth disease virus green vaccine candidate based on plant-produced recombinant protein (1 project)

[Contribution Ratio] 80/100

[Name of Project Executing Organization] Pohang University of Science and Technology

[Research Period] January 1, 2021 to Dec. 31, 2021

(2) [Project Identification Number] 1345333243

[Project Number] 2021R1I1A1A01051391

[Name of Ministry] Ministry of Education

[Name of Project Management (Specialized) Organization] National Research Foundation of Korea

[Research Project Name] Establishment of science and engineering academic research base

[Research Task Name] Induction of high expression of plant-based recombinant insulin and development of protein production system

[Contribution Ratio] 20/100

[Name of Project Executing Organization] Pohang University of Science and Technology

[Research Period] June 1, 2021 to Feb. 28, 2022

1-24. (canceled)

25. A gene construct for high expression of a gene of a protein of interest, comprising an FMM-UD promoter in which the following (i) and (ii) are sequentially linked:
(i) an FMM promoter in which a Figwort subgenomic transcript promoter gene fragment, a Mirabilis mosaic virus subgenomic transcript promoter gene fragment and a Mirabilis mosaic virus full-length transcript promoter gene fragment are sequentially linked; and
(ii) an upstream DNA (UD) sequence with 4 repetitions of an upstream activation sequence (UAS), which is a GAL4-binding site of yeast, linked to a 5′ end of the FMM promoter.

26. The gene construct of claim 25, wherein the Figwort subgenomic transcript promoter gene comprises a nucleotide sequence of SEQ ID NO: 1, the Mirabilis mosaic virus subgenomic transcript promoter gene comprises a nucleotide sequence of SEQ ID NO: 2, and the Mirabilis mosaic virus full-length transcript promoter gene comprises a nucleotide sequence of SEQ ID NO: 3.

27. The gene construct of claim 25, wherein the FMM-UD promoter gene comprises a nucleotide sequence of SEQ ID NO: 4.

28. The gene construct of claim 25, wherein a TATA box sequence is comprised at a 3′ end of the FMM-UD promoter, and a 5′ UTR gene is linked to a 5′ end of the FMM-UD promoter.

29. The gene construct of claim 28, wherein the 5′ UTR gene comprises a nucleotide sequence of SEQ ID NO: 5.

30. The gene construct of claim 25, further comprising a 3PR terminator in which a cauliflower mosaic virus 35S terminator gene, a 3′ region of a potato proteinase inhibitor II gene, and a gene of an RB7 scaffold attachment site are sequentially linked.

31. The gene construct of claim 30, wherein the gene construct comprises an expression cassette comprising a chaperone binding protein (BiP) gene, a mannosylated peptide region (MP) gene, a cellulose-binding module 3 (CBM3) gene, a small ubiquitin-related modifier (SUMO) gene, a gene encoding a protein of interest and a gene encoding His-Asp-Glu-Leu (HDEL) between the FMM-UD promoter and the 3PR terminator.

32. The gene construct of claim 31, wherein an amino acid sequence of the BiP comprises an amino acid sequence of SEQ ID NO: 11, an amino acid sequence of the MP comprises an amino acid sequence of SEQ ID NO: 13, an amino acid sequence of the CBM3 comprises an amino acid sequence of SEQ ID NO: 17, and an amino acid sequence of the SUMO comprises an amino acid sequence of SEQ ID NO: 19.

33. The gene construct of claim 31, wherein the BiP gene comprises a nucleotide sequence of SEQ ID NO: 10, the MP gene comprises a nucleotide sequence of SEQ ID NO: 12, the CBM3 gene comprises a nucleotide sequence of SEQ ID NO: 16, and the SUMO gene comprises a nucleotide sequence of SEQ ID NO: 18.

34. A gene construct for high expression of a gene of a protein of interest, comprising a 3PR terminator in which a cauliflower mosaic virus 35S terminator gene, a 3′ region of a potato proteinase inhibitor II gene, and a gene of an RB7 scaffold attachment site are sequentially linked.

35. The gene construct of claim 34, wherein the cauliflower mosaic virus 35S terminator gene comprises a nucleotide sequence of SEQ ID NO: 6, the 3′ region of the potato proteinase inhibitor II gene comprises a nucleotide sequence of SEQ ID NO: 7, and the gene of the RB7 scaffold attachment site comprises a nucleotide sequence of SEQ ID NO: 8.

36. The gene construct of claim 34, wherein the 3PR terminator comprises a nucleotide sequence of SEQ ID NO: 9.

37. A recombinant expression vector for high expression of a gene of a protein of interest, comprising:
the gene construct according to claim 25; and
a gene encoding a protein of interest.

38. A recombinant expression vector for high expression of a gene of a protein of interest, comprising:
the gene construct according to claim 34; and
a gene encoding a protein of interest.

39. The recombinant expression vector of claim 37, wherein the protein of interest is at least any one protein selected from the group consisting of human interleukin 6, a transcription factor, a toxic protein, a hormone, a hormone analog, a cytokine, a movement protein, an enzyme, an enzyme inhibitor, a transport protein, a structural protein, a receptor, a receptor fragment, a biological defense inducer, a storage protein, an exploitative protein, a reporter protein, an antigen, an antibody and an antibody fragment.

40. The recombinant expression vector of claim 38, wherein the protein of interest is at least any one protein selected from the group consisting of human interleukin 6, a transcription factor, a toxic protein, a hormone, a hormone analog, a cytokine, a movement protein, an enzyme, an enzyme inhibitor, a transport protein, a structural protein, a receptor, a receptor fragment, a biological defense inducer, a storage protein, an exploitative protein, a reporter protein, an antigen, an antibody and an antibody fragment.

41. A transformant for high expression of a gene of a protein of interest, transformed with the recombinant expression vector of claim 37.

42. A transformant for high expression of a gene of a protein of interest, transformed with the recombinant expression vector of claim 38.

43. A plant, into which the transformant of claim 41 is introduced.

44. A plant, into which the transformant of claim 42 is introduced.
